SEQUENCE LISTING (15 sequences)

SEQ ID NO: 1 - DNA, 23 bp, synthetic construct
  source 1..23: mol_type = other DNA
cttctaaccgaggtcgaaacgta

SEQ ID NO: 2 - DNA, 23 bp, synthetic construct
  source 1..23: mol_type = other DNA
cttcttaccgaggtcgaaacgta

SEQ ID NO: 3 - DNA, 20 bp, synthetic construct
  source 1..20: mol_type = other DNA
agagcctcaagatctgtgtt

SEQ ID NO: 4 - DNA, 24 bp, synthetic construct
  source 1..24: mol_type = other DNA
tgtcgctgtttggagacacaattg

SEQ ID NO: 5 - DNA, 20 bp, synthetic construct
  source 1..20: mol_type = other DNA
ttctttcccaccgaaccaac

SEQ ID NO: 6 - DNA, 20 bp, synthetic construct
  source 1..20: mol_type = other DNA
ttctttcccaccaaaccaac

SEQ ID NO: 7 - DNA, 19 bp, synthetic construct
  source 1..19: mol_type = other DNA
agatttggacctgcgagcg

SEQ ID NO: 8 - DNA, 19 bp, synthetic construct
  source 1..19: mol_type = other DNA
caaggtgagcggctgtctc

SEQ ID NO: 9 - DNA, 21 bp, synthetic construct
  source 1..21: mol_type = other DNA
tcaggccccctcaaagccgag

SEQ ID NO: 10 - DNA, 30 bp, synthetic construct
  source 1..30: mol_type = other DNA
agaagatggagaaggcaaagcagaactagc

SEQ ID NO: 11 - DNA, 23 bp, synthetic construct
  source 1..23: mol_type = other DNA
ttctgacctgaaggctctgcgcg

SEQ ID NO: 12 - DNA, 119 bp, synthetic construct
  source 1..119: mol_type = other DNA
cttctaaccgaggtcgaaacgtatgttctctctatcgttccatcaggccccctcaaagccgagatcgcgcagagacttgaagatgtctttgctgggaaaaacacagatcttgaggctct

SEQ ID NO: 13 - DNA, 107 bp, synthetic construct
  source 1..107: mol_type = other DNA
tgtcgctgtttggagacacaattgcctacctgctttcactaatagaagatggagaaggcaaagcagaactagctgaaaaattacactgttggttcggtgggaaagaa

SEQ ID NO: 14 - DNA, 71 bp, Homo sapiens
  source 1..71: mol_type = genomic DNA
agatttggacctgcgagcgggttctgacctgaaggctctgcgcggacttgtggagacagccgctcaccttg

SEQ ID NO: 15 - DNA, 24 bp, synthetic construct
  source 1..24: mol_type = other DNA
catgagagcctcaagatctgtgtt

(19) United States
(12) Patent Application Publication
(10) Pub. No.: US 2025/0297330 A1
(43) Pub. Date: Sep. 25, 2025

(54) METHOD AND KIT FOR DETECTING INFLUENZA A AND B VIRUSES

(71) Applicant: DELTA ELECTRONICS, INC., Taoyuan City (TW)

(72) Inventors: Yu-Han SHEN, Taoyuan City (TW); Wei-Fan CHEN, Taoyuan City (TW); Ying-Ting CHEN, Taoyuan City (TW)

(21) Appl. No.: 18/779,108

(22) Filed: Jul. 22, 2024

(30) Foreign Application Priority Data
Mar. 19, 2024 (CN) 202410311030.8

(51) Int. Cl. C12Q 1/70

(52) U.S. Cl. CPC C12Q 1/701; C12Q 2600/16

(57) ABSTRACT

A method and a kit for detecting influenza A and B viruses provides a primer group that can simultaneously detect influenza A virus and influenza B virus, in which a first primer pair uses a specific genome fragment of influenza A virus as an amplification target, and a second primer pair uses a specific genome fragment of influenza B virus as an amplification target. The primer group can withstand at least 300 nanograms (ng) of human genomic DNA interference in reverse transcription polymerase chain reaction when detecting influenza A and B viruses, thereby making detection sensitivity less susceptible to interference from human genomic DNA in the sample.

### CROSS-REFERENCE TO RELATED APPLICATION

This application claims priority to China Application Serial Number 202410311030.8, filed Mar. 19, 2024, which is herein incorporated by reference in its entirety.

The Sequence Listing associated with this application is filed in electronic format via EFS-Web and is hereby incorporated by reference into the specification in its entirety. The name of the XML file containing the Sequence Listing is NP-36122-US_SEQ_LIST.xml. The size of the XML file is 14,507 bytes, and the XML file was created on Jul. 8, 2024.

### BACKGROUND

### Field of Invention

The present disclosure relates to a method and a kit thereof. More particularly, the present disclosure relates to a method and a kit for detecting influenza A and B viruses.

### Description of Related Art

Influenza virus belongs to Orthomyxoviridae, and can be divided into four types: A, B, C, and D according to classification of nucleocapsid protein (NP) and membrane protein (MP), and only types A, B, and C can infect humans, and type D has only been found in pigs and cows. Influenza A virus can be further divided into multiple subtypes based on differences in two proteins: hemagglutinin (HA) and neuraminidase (NA), represented by numbers, such as H1N1, H3N2, etc. Type B is divided into two lineages, Yamagata and Victoria, based on antigenicity.

At present, main methods for detecting influenza viruses include virus cultivation, serological detection (including a rapid screening reagent), and viral nucleic acid detection. Among them, the virus cultivation takes more than two days. Although the rapid screening reagent only takes tens of minutes, it has a problem of false negatives due to insufficient sensitivity. Doctors still need to consider clinical symptoms whether to prescribe antiviral drugs. Only the method of nucleic acid detection can meet requirements of both speed and sensitivity.

Therefore, the related art really needs to be improved.

### SUMMARY

One embodiment of the present disclosure provides a method for detecting influenza A and B viruses, which includes: providing a sample; providing a first primer pair, in which a first forward primer of the first primer pair is a nucleotide sequence, its complementary nucleotide sequence or its derivative sequence selected from the group consisting of SEQ ID NO: 1 and a sequence that is from 80% to about 99% identical to SEQ ID NO: 1; and a second forward primer of the first primer pair is a nucleotide sequence, its complementary nucleotide sequence or its derivative sequence selected from the group consisting of SEQ ID NO: 2 and a sequence that is from 80% to about 99% identical to SEQ ID NO: 2; and a reverse primer of the first primer pair is a nucleotide sequence, its complementary nucleotide sequence or its derivative sequence selected from the group consisting of SEQ ID NO: 3 and a sequence that is from 80% to about 99% identical to SEQ ID NO: 3; providing a second primer pair, in which a forward primer of the second primer pair is a nucleotide sequence, its complementary nucleotide sequence or its derivative sequence selected from the group consisting of SEQ ID NO: 4 and a sequence that is from 80% to about 99% identical to SEQ ID NO: 4; and a first reverse primer of the second primer pair is a nucleotide sequence, its complementary nucleotide sequence or its derivative sequence selected from the group consisting of SEQ ID NO: 5 and a sequence that is from 80% to about 99% identical to SEQ ID NO: 5; and a second reverse primer of the second primer pair is a nucleotide sequence, its complementary nucleotide sequence or its derivative sequence selected from the group consisting of SEQ ID NO: 6 and a sequence that is from 80% to about 99% identical to SEQ ID NO: 6; and performing a polymerase chain reaction on the sample using the first primer pair and the second primer pair, in which when a product obtained through the polymerase chain reaction includes two with 90 to 130 base pairs, the influenza A and B viruses are detected; or when the product is not present, the influenza A and B viruses are not detected.

In some embodiments, the sample is nasal mucus, saliva, sputum, blood, urine, feces or a combination thereof.

In some embodiments, the step of performing the polymerase chain reaction on the sample using the first primer pair and the second primer pair includes: performing the polymerase chain reaction such that the first primer pair amplifies a portion of a sequence of the influenza A virus to SEQ ID NO: 12, and performing the polymerase chain reaction such that the second primer pair amplifies a portion of a sequence of the influenza B virus to SEQ ID NO: 13.

In some embodiments, a concentration ratio of the forward primer to the reverse primer of the first primer pair is from 1:0.5 to 1:2.

In some embodiments, a concentration ratio of the forward primer to the reverse primer of the second primer pair is from 1:1 to 1:2.

In some embodiments, a concentration ratio of the first reverse primer to the second reverse primer of the reverse primer of the second primer pair is from 1:1 to 1:2.

In some embodiments, the method further includes: providing a first probe, which is a nucleotide sequence, its complementary nucleotide sequence or its derivative sequence selected from the group consisting of SEQ ID NO: 9 and a sequence that is from 80% to about 99% identical to SEQ ID NO: 9; providing a second probe, which is a nucleotide sequence, its complementary nucleotide sequence or its derivative sequence selected from the group consisting of SEQ ID NO: 10 and a sequence that is from 80% to about 99% identical to SEQ ID NO: 10; and performing the polymerase chain reaction on the sample using the first primer pair, the second primer pair, the first probe and the second probe.

In some embodiments, the polymerase chain reaction is a real-time polymerase chain reaction.

In some embodiments, the method further includes providing a third primer pair, in which a forward primer of the third primer pair is a nucleotide sequence, its complementary nucleotide sequence or its derivative sequence selected from the group consisting of SEQ ID NO: 7 and a sequence that is from 80% to about 99% identical to SEQ ID NO: 7; a reverse primer of the third primer pair is a nucleotide sequence, its complementary nucleotide sequence or its derivative sequence selected from the group consisting of SEQ ID NO: 8 and a sequence that is from 80% to about 99% identical to SEQ ID NO: 8; and performing the polymerase chain reaction on the sample using the first primer pair, the second primer pair and the third primer pair, in which when the product obtained through the polymerase chain reaction further includes one with 60 to 80 base pairs, the sample is confirmed from a human.

In some embodiments, the step of performing the polymerase chain reaction on the sample using the first primer pair, the second primer pair and the third primer pair includes: performing the polymerase chain reaction such that the third primer pair amplifies a portion of a sequence of ribonuclease P of the human to SEQ ID NO: 14.

In some embodiments, the method further includes providing a first probe, which is a nucleotide sequence, its complementary nucleotide sequence or its derivative sequence selected from the group consisting of SEQ ID NO: 9 and a sequence that is from 80% to about 99% identical to SEQ ID NO: 9; providing a second probe, which is a nucleotide sequence, its complementary nucleotide sequence or its derivative sequence selected from the group consisting of SEQ ID NO: 10 and a sequence that is from 80% to about 99% identical to SEQ ID NO: 10; providing a third probe, which is a nucleotide sequence, its complementary nucleotide sequence or its derivative sequence selected from the group consisting of SEQ ID NO: 11 and a sequence that is from 80% to about 99% identical to SEQ ID NO: 11; and performing the polymerase chain reaction on the sample using the first primer pair, the second primer pair, the third primer pair, the first probe, the second probe and the third probe.

The present disclosure also provides a kit for detecting influenza A and B viruses, which includes: a first primer pair and a second primer pair. A first forward primer of the first primer pair is a nucleotide sequence, its complementary nucleotide sequence or its derivative sequence selected from the group consisting of SEQ ID NO: 1 and a sequence that is from 80% to about 99% identical to SEQ ID NO: 1; and a second forward primer of the first primer pair is a nucleotide sequence, its complementary nucleotide sequence or its derivative sequence selected from the group consisting of SEQ ID NO: 2 and a sequence that is from 80% to about 99% identical to SEQ ID NO: 2; and a reverse primer of the first primer pair is a nucleotide sequence, its complementary nucleotide sequence or its derivative sequence selected from the group consisting of SEQ ID NO: 3 and a sequence that is from 80% to about 99% identical to SEQ ID NO: 3. A forward primer of the second primer pair is a nucleotide sequence, its complementary nucleotide sequence or its derivative sequence selected from the group consisting of SEQ ID NO: 4 and a sequence that is from 80% to about 99% identical to SEQ ID NO: 4; and a first reverse primer of the second primer pair is a nucleotide sequence, its complementary nucleotide sequence or its derivative sequence selected from the group consisting of SEQ ID NO: 5 and a sequence that is from 80% to about 99% identical to SEQ ID NO: 5; and a second reverse primer of the second primer pair is a nucleotide sequence, its complementary nucleotide sequence or its derivative sequence selected from the group consisting of SEQ ID NO: 6 and a sequence that is from 80% to about 99% identical to SEQ ID NO: 6.

In some embodiments, the method further includes a first probe and a second probe. The first probe is a nucleotide sequence, its complementary nucleotide sequence or its derivative sequence selected from the group consisting of SEQ ID NO: 9 and a sequence that is from 80% to about 99% identical to SEQ ID NO: 9. The second probe is a nucleotide sequence, its complementary nucleotide sequence or its derivative sequence selected from the group consisting of SEQ ID NO: 10 and a sequence that is from 80% to about 99% identical to SEQ ID NO: 10.

In some embodiments, the method further includes a third primer pair, in which a forward primer of the third primer pair is a nucleotide sequence, its complementary nucleotide sequence or its derivative sequence selected from the group consisting of SEQ ID NO: 7 and a sequence that is from 80% to about 99% identical to SEQ ID NO: 7; and a reverse primer of the third primer pair is a nucleotide sequence, its complementary nucleotide sequence or its derivative sequence selected from the group consisting of SEQ ID NO: 8 and a sequence that is from 80% to about 99% identical to SEQ ID NO: 8.

In some embodiments, the method further includes a third probe, which is a nucleotide sequence, its complementary nucleotide sequence or its derivative sequence selected from the group consisting of SEQ ID NO: 11 and a sequence that is from 80% to about 99% identical to SEQ ID NO: 11.

### BRIEF DESCRIPTION OF THE DRAWINGS

Aspects of the present disclosure are best understood from the following detailed description when read with the accompanying figures. It is noted that, in accordance with the standard practice in the industry, various features may be not drawn to scale. In fact, dimensions of the various features may be arbitrarily increased or reduced for clarity of discussion. The disclosure can be more fully understood by reading the following detailed description of the embodiments, with reference made to the accompanying drawings as follows:

FIG. 1 illustrates a partial sequence diagram and a schematic diagram of design positions of primers and a probe of influenza A virus matrix protein (M) gene according to one embodiment of the present disclosure.

FIG. 2 illustrates a partial sequence diagram and a schematic diagram of design positions of primers and a probe of influenza B virus matrix protein gene according to one embodiment of the present disclosure.

FIG. 3 illustrates a partial sequence diagram and a schematic diagram of design positions of primers and a probe of human gene ribonuclease P (RNase P) according to one embodiment of the present disclosure.

FIG. 4 is a standard curve diagram of different template starting quantities for influenza A virus matrix protein gene according to one embodiment of the present disclosure (IVT is the abbreviation of in-vitro transcription, RNA synthesized in vitro). FIGS. 6 and 8 below are also same.

FIG. 5 is a standard curve and an R-squared value of FIG. 4.

FIG. 6 is a standard curve diagram of different template starting quantities for influenza B virus matrix protein gene according to one embodiment of the present disclosure.

FIG. 7 is a standard curve and an R-squared value of FIG. 6.

FIG. 8 is a standard curve diagram of different template starting quantities for human gene ribonuclease P according to one embodiment of the present disclosure.

FIG. 9 is a standard curve and an R-squared value of FIG. 8.

FIG. 10 is an electrophoresis result diagram of detections of influenza A virus matrix protein gene, influenza B virus matrix protein gene and human gene ribonuclease P according to one embodiment of the present disclosure.

FIG. 11 illustrates a partial sequence diagram and a schematic diagram of design positions of primers and a probe of influenza A virus matrix protein gene according to another embodiment of the present disclosure.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

Further, when a number or a range of numbers is described with “about,” “approximate,” and the like, the term is intended to encompass numbers that are within a reasonable range considering variations that inherently arise during manufacturing as understood by one of ordinary skill in the art. For example, the number or range of numbers encompasses a reasonable range including the number described, such as within +/−10% of the number described, based on known manufacturing tolerances associated with manufacturing a feature having a characteristic associated with the number.

Embodiments of the present disclosure provides a method and a kit for detecting influenza A and B viruses, which provides a primer group that can simultaneously detect influenza A virus (Flu A) and influenza B virus (Flu B). The first primer pair uses a specific genome fragment of influenza A virus as an amplification target, and a second primer pair uses a specific genome fragment of influenza B virus as an amplification target. The primer group can withstand at least 300 nanograms (ng) of human genomic DNA interference in reverse transcription polymerase chain reaction when detecting influenza A and B viruses, thereby making detection sensitivity less susceptible to interference from the human genomic DNA.

Embodiments of the present disclosure provide a method for detecting influenza A and B viruses, which includes following steps: providing a sample; providing a first primer pair, in which a first forward primer of the first primer pair is a nucleotide sequence, its complementary nucleotide sequence or its derivative sequence selected from the group consisting of SEQ ID NO: 1 and a sequence that is from 80% to about 99% identical to SEQ ID NO: 1; and a second forward primer of the first primer pair is a nucleotide sequence, its complementary nucleotide sequence or its derivative sequence selected from the group consisting of SEQ ID NO: 2 and a sequence that is from 80% to about 99% identical to SEQ ID NO: 2; and a reverse primer of the first primer pair is a nucleotide sequence, its complementary nucleotide sequence or its derivative sequence selected from the group consisting of SEQ ID NO: 3 and a sequence that is from 80% to about 99% identical to SEQ ID NO: 3; providing a second primer pair, in which a forward primer of the second primer pair is a nucleotide sequence, its complementary nucleotide sequence or its derivative sequence selected from the group consisting of SEQ ID NO: 4 and a sequence that is from 80% to about 99% identical to SEQ ID NO: 4; and a first reverse primer of the second primer pair is a nucleotide sequence, its complementary nucleotide sequence or its derivative sequence selected from the group consisting of SEQ ID NO: 5 and a sequence that is from 80% to about 99% identical to SEQ ID NO: 5; and a second reverse primer of the second primer pair is a nucleotide sequence, its complementary nucleotide sequence or its derivative sequence selected from the group consisting of SEQ ID NO: 6 and a sequence that is from 80% to about 99% identical to SEQ ID NO: 6; and performing a polymerase chain reaction on the sample using the first primer pair and the second primer pair, in which when a product obtained through the polymerase chain reaction includes two with 90 to 130 base pairs, the influenza A and B viruses are detected; or when the product is not present, the influenza A and B viruses are not detected.

In some embodiments, the sample may include specimens from various sources, such as nasal mucus, saliva, sputum, blood, urine, feces or a combination thereof.

The term “nasal mucus” (also known as snot, nasal discharge) refers to the mucus in the nose. Nasal mucus is secreted by goblet cells of nasal mucosa, and its function is to protect the respiratory tract.

In some embodiments, the specimen provided in the method for detecting influenza A and B viruses includes a portion of a sequence of influenza A virus as SEQ ID NO: 12 and a portion of a sequence of influenza B virus as SEQ ID NO: 13.

In some embodiments, selections of the primer pairs are as described above and are not limited to SEQ ID NO: 1, 2 and 3 and SEQ ID NO: 4, 5 and 6 disclosed herein. In addition to complementary strands of SEQ ID NO: 1, 2, and 3 and complementary strands of SEQ ID NO: 4, 5, and 6, sequences shown in SEQ ID NO: 1, 2, 3, and SEQ ID NO: 4, 5, and 6 may also allow a certain degree of variation. That is, sequences that are about 80% to about 99% identical to SEQ ID NO: 1, 2, and 3 and sequences that are about 80% to about 99% identical to SEQ ID NO: 4, 5, and 6 also have the same effect when used in the embodiments. For example, the selections of the primer pairs may include degenerate sequences of SEQ ID NO: 1, 2, and 3 and degenerate sequences of SEQ ID NO: 4, 5, and 6. The term “degenerate sequence” herein refers to an oligonucleotide sequence disclosed herein in which some nucleotides are replaced by other nucleotides. In other words, the degenerate sequences of SEQ ID NO: 1, 2, and 3 mean that oligonucleotides can be allowed to have variations of about 1% to about 20% while sequence lengths of SEQ ID NO: 1, 2, and 3 remain unchanged. The degenerate sequences of SEQ ID NO: 4, 5, and 6 mean that oligonucleotides can be allowed to have variations of about 1% to about 20% while sequence lengths of SEQ ID NO: 4, 5, and 6 remain unchanged. In other embodiments, the selections of the primer pairs may also include derivative sequences of SEQ ID NO: 1, 2, and 3 and derivative sequences of SEQ ID NO: 4, 5, and 6. The “derivative sequence” herein refers to an oligonucleotide sequence disclosed herein that can be modified at the 3′ end or the 5′ end and still retains a portion or all of the sequence. In other words, the derivative sequences of SEQ ID NO: 1, 2, and 3 mean that oligonucleotides of SEQ ID NO: 1, 2, and 3 can be allowed to have variations of about 1% to about 20% while sequence lengths of SEQ ID NO: 1, 2, and 3 can be increased or decreased. The derivative sequences of SEQ ID NO: 4, 5, and 6 mean that oligonucleotides of SEQ ID NO: 4, 5, and 6 can be allowed to have variations of about 1% to about 20% while sequence lengths of SEQ ID NO: 4, 5, and 6 can be increased or decreased. In other embodiments, the first primer pair is selected from sequences that are about 80% to about 99% identical to SEQ ID NO: 1, 2, 3 (e.g., 82%, 84%, 86%, 88%, 90%, 92%, 94%, 96%, 98%, or any value between any two of these values), and the second primer pair is selected from sequences that are about 80% to about 99% identical to SEQ ID NO: 4, 5, 6 (e.g., 82%, 84%, 86%, 88%, 90%, 92%, 94%, 96%, 98%, or any value between any two of these values).

In some embodiments, a concentration ratio of the forward primer to the reverse primer of the first primer pair is from 1:0.5 to 1:2, such as 1:0.6, 1:0.7, 1:0.8, 1:0.9, 1:1.0, 1:1.1, 1:1.2, 1:1.3, 1:1.4, 1:1.5, 1:1.6, 1:1.7, 1:1.8, 1:1.9, or any value between any two of these values.

In some embodiments, a concentration ratio of the forward primer to the reverse primer of the second primer pair is from 1:1 to 1:2, such as 1:1.1, 1:1.2, 1:1.3, 1:1.4, 1:1.5, 1:1.6, 1:1.7, 1:1.8, 1:1.9, or any value between any two of these values.

In some embodiments, a concentration ratio of the first reverse primer to the second reverse primer of the second primer pair is from 1:1 to 1:2, for example, 1:1.1, 1:1.2, 1:1.3, 1:1.4, 1:1.5, 1:1.6, 1:1.7, 1:1.8, 1:1.9, or any value between any two of these values.

In some embodiments, selections of the first probe and the second probe are as described above and are not limited to SEQ ID NO: 9 and 10 disclosed herein. In the selections of the first probe and the second probe, in addition to complementary strands of SEQ ID NO: 9 and 10, sequences shown in SEQ ID NO: 9 and 10 may also allow a certain degree of variation. In other words, sequences with a similarity of about 80% to about 99% to SEQ ID NO: 9 and 10 also have the same effect when used in the embodiments. For example, the selections of the first probe and the second probe may include degenerate sequences of SEQ ID NO: 9 and 10. The degenerate sequences of SEQ ID NO: 9 and 10 mean that oligonucleotides of SEQ ID NO: 9 and 10 can be allowed to have variations of about 1% to about 20% while sequence lengths of SEQ ID NO: 9 and 10 remain unchanged. In other embodiments, the selections of the first probe and the second probe may also include derivative sequences of SEQ ID NO: 9 and 10. The derivative sequences of SEQ ID NO: 9 and 10 mean that oligonucleotides thereof can be allowed to have variations of about 1% to about 20% while sequence lengths of SEQ ID NO: 9 and 10 are increased or decreased at the 3′ end or 5′ end. In other embodiments, the first probe and the second probe are selected from sequences with about 80% to about 99% similarity to SEQ ID NO: 9, 10 (e.g., 82%, 84%, 86%, 88%, 90%, 92%, 94%, 96%, 98%, or any value between any two of these values). In some embodiments, the probes include, but are not limited to, oligonucleotide probes.

In some embodiments, performing a polymerase chain reaction on the sample using the first primer pair, the second primer pair, the first probe and the second probe to obtain a product includes: performing the polymerase chain reaction such that the first primer pair amplifies a portion of a sequence of the influenza A virus to SEQ ID NO: 12 and performing the polymerase chain reaction such that the second primer pair amplifies a portion of a sequence of the influenza B virus to SEQ ID NO: 13 to obtain the product. The polymerase chain reaction is a molecular biology technique. The primer pair having the oligonucleotide sequence is used to amplify a specific deoxyribonucleic acid (DNA) fragment. It will be appreciated that the sequences disclosed herein may be used in a variety of polymerase chain reaction-based techniques. In some embodiments, the polymerase chain reaction may include, but is not limited to, real-time polymerase chain reaction (real-time PCR). In some embodiments, when the real-time polymerase chain reaction used is a probe-type fluorescent system, a hybridization reaction is performed on the sample using the first probe and the second probe to let the probes bind to target sequences before performing the polymerase chain reaction on the sample using the first primer pair and the second primer pair to obtain the product. That is, the first primer pair, the second primer pair, the first probe, the second probe and the sample perform the polymerase chain reaction together to obtain the product and fluorescent signals.

In other embodiments, the method for detecting influenza A and B viruses further includes providing a third primer pair, in which a forward primer of the third primer pair is a nucleotide sequence, its complementary nucleotide sequence or its derivative sequence selected from the group consisting of SEQ ID NO: 7 and a sequence that is from 80% to about 99% identical to SEQ ID NO: 7; a reverse primer of the third primer pair is a nucleotide sequence, its complementary nucleotide sequence or its derivative sequence selected from the group consisting of SEQ ID NO: 8 and a sequence that is from 80% to about 99% identical to SEQ ID NO: 8; performing the polymerase chain reaction on the sample using the first primer pair, the second primer pair and the third primer pair, in which when the product obtained through the polymerase chain reaction further comprises one with 60 to 80 base pairs, it is confirmed that the sample is from a human.

In some embodiments, when the specimen source provided in the method for detecting influenza A and B viruses is a human, the step of performing the polymerase chain reaction on the sample using the first primer pair, the second primer pair and the third primer pair includes: performing the polymerase chain reaction such that the third primer pair amplifies a portion of a sequence of a ribonuclease P of the human to SEQ ID NO: 14.

In some embodiments, the description of the nucleotide sequences, their complementary nucleotide sequences or their derivative sequences selected from the group consisting of sequences with 80% to about 99% identity of the third primer pair is also as described above. The third primer pair is selected from sequences that are about 80% to about 99% identical to SEQ ID NO: 7, 8 (e.g., 82%, 84%, 86%, 88%, 90%, 92%, 94%, 96%, 98%, or any value between any two of these values).

In some embodiments, a third probe is a nucleotide sequence, its complementary nucleotide sequence or its derivative sequence selected from the group consisting of SEQ ID NO: 11 and a sequence that is from 80% to about 99% identical to SEQ ID NO: 11, which is also as described above. The third primer pair is selected from a sequence that is about 80% to about 99% identical to SEQ ID NO: 11 (e.g., 82%, 84%, 86%, 88%, 90%, 92%, 94%, 96%, 98%, or any value between any two of these values).

Embodiments of the present disclosure also provides a kit for detecting influenza A and B viruses, which includes a first primer pair and a second primer pair as described above. In some embodiments, the kit further includes a third primer pair as described above to confirm that the sample is from a human.

In some embodiments, the kit further includes a first probe, a second probe, and a third probe as described above, which are provided for use in real-time polymerase chain reaction.

In some embodiments, the kit for detecting influenza A and B viruses may further include a specimen. The source of the specimen can be nasal mucus, saliva, sputum, blood, urine, feces or a combination thereof. For example, the kit for detecting influenza A and B viruses may be used in various medical units by collecting nasal mucus, saliva or sputum from individuals (e.g., human) for detection.

Several embodiments and experimental examples are listed below to further elaborate on the method and kit for detecting influenza A and B viruses of the present disclosure. However, they are only for illustrative purposes and are not intended to limit the present disclosure. The protection scope of the present disclosure shall be defined by the appended claims.

### EXAMPLES

Although a series of operations or steps are used below to describe the method disclosed herein, an order of these operations or steps should not be construed as a limitation to the present disclosure. For example, some operations or steps may be performed in a different order and/or other steps may be performed at the same time. In addition, all shown operations, steps and/or features are not required to be executed to implement an embodiment of the present disclosure. In addition, each operation or step described herein may include a plurality of sub-steps or actions.

For the sake of clarity, features and elements that are known in the art and are not necessary for an understanding of the principles described have been omitted.

Examples of the present disclosure utilized real-time reverse transcription polymerase chain reaction (referred to as real-time reverse transcription-PCR) to instantly detect specific fragments of nucleic acid molecules in the specimen, in which influenza A virus matrix protein gene, influenza B virus matrix protein gene and human gene ribonuclease P were used as amplification targets in the present disclosure to achieve rapid and highly sensitive diagnosis of influenza virus-infected specimens. In addition, each primer group and probe could withstand interference of high-concentration human genomic DNA and achieve ability to withstand the interference of genomic DNA in nucleic acid extraction products. The real-time reverse transcription-PCR mainly included three steps. In the first step, the nucleic acid molecule RNA of influenza A, the nucleic acid molecule RNA of influenza B, and the nucleic acid molecule RNA of human gene ribonuclease P were reverse-transcribed into complementary DNAs. Next, in the second step, the polymerase chain reaction was used to amplify specific fragments of nucleic acid molecules. In the third step, a reporter system dye on a real-time detection probe was used to achieve a purpose of real-time detection. The 5′ end of the reporter dye of the probe (including, but not limited to, FAM, ROX, HEX, SUN, etc.) and the 3′ quencher (including, but not limited to, BHQ1, BHQ2, BHQ3, etc.) would be separated during real-time reverse transcription-PCR, and separation of the reporter gene and the quencher would emit fluorescent signals in the reaction tube. The present disclosure provides a method for detecting various mutant strains of influenza A and B viruses while maintaining a sequence coverage rate of more than or equal to 90%.

### Example 1

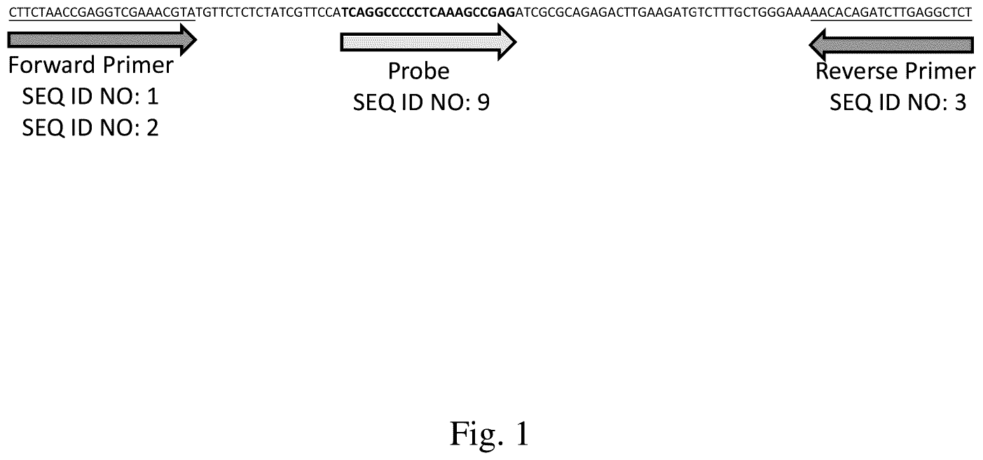

Please refer to FIGS. 1 to 3, which illustrate partial sequence diagrams of influenza A virus matrix protein gene, influenza B virus matrix protein gene and human gene ribonuclease P and schematic diagrams of design positions of primers and probes according to one embodiment of the present disclosure. Matrix protein gene sequences of influenza A and B viruses were highly conserved. Therefore, the present disclosure designs the primers and probes based on the matrix protein gene sequences of influenza A and B viruses.

Influenza A is a single-stranded RNA virus with a total genome of 13,627 bases (influenza A virus strain H3N2, referenced from NCBI Taxonomy ID: 335341). In the present disclosure, the matrix protein (M) gene of influenza A virus was one of detection targets. FIG. 1 illustrates a partial sequence of influenza A matrix protein gene (corresponding to positions 7 to 125 of sequence AB704433.1, SEQ ID NO: 12) and positions where forward primers, a reverse primer and a probe were bound to the gene sequence, and the forward primers are shown in SEQ ID NO: 1 and 2 and start from the 7th base position of the matrix protein gene with a length of 23 bases (mer); the reverse primer is shown in SEQ ID NO: 3 and starts from the 125th base position of the matrix protein gene with a length of 20 mer; the probe is a forward probe shown in SEQ ID NO: 9 and starts from the 49th position of the matrix protein gene with a length of 21 mer. It could be amplified to form an amplification product with a size of 119 mer using the forward primers and the reverse primer mentioned above. The primer pair and probe combination designed in the present disclosure to detect influenza A had a coverage rate of >90% for NCBI sequence comparisons from 2018 to October 2023 (Table 1 below), and no identical sequence fragments were found through comparisons of other similar species. It showed that specificity of the primer pair and probe combination for detecting influenza A was very high.

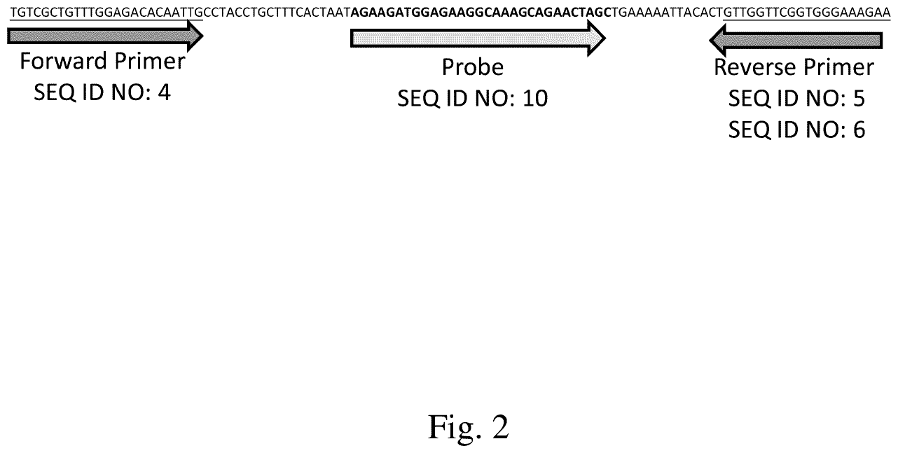

Influenza B of the present disclosure is a single-stranded RNA virus with a total genome of 14,452 bases (influenza B virus strain B/Lee/1940, referenced from NCBI Taxonomy ID: 518987). In the present disclosure, the matrix protein gene of influenza B was one of the detection targets. FIG. 2 illustrates a partial sequence of influenza B matrix protein gene (corresponding to positions 15 to 110 of sequence MT243910.1, SEQ ID NO: 13) and positions where a forward primer, reverse primers and a probe were bound to the gene sequence. The forward primer is shown in SEQ ID NO: 4 and starts from the 15th base position of the matrix protein gene with a length of 24 mer; the reverse primers are shown in SEQ ID NO: 5 and 6 and start from the 110th base position of the matrix protein gene with a length of 20 mer; the probe is a forward probe shown in SEQ ID NO: 10 and starts from the 58th position of the matrix protein gene with a length of 30 mer. It could be amplified to form an amplification product with a size of 107 mer using the forward primer and the reverse primers mentioned above. The primer pair and probe combination designed in the present disclosure to detect influenza B had a coverage rate of >97% for NCBI sequence comparisons from 2018 to October 2023 (Table 1 below), and no identical sequence fragments were found through comparisons of other similar species. It showed that specificity of the primer pair and probe combination for detecting influenza B was very high.

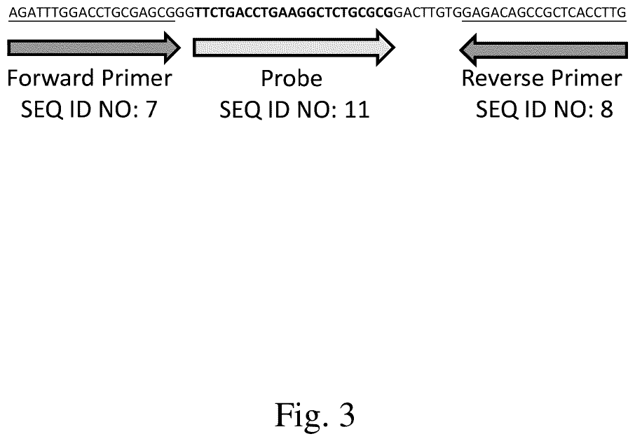

Human gene ribonuclease P of the present disclosure was the third detection target. FIG. 3 illustrates a partial sequence of human gene ribonuclease P (corresponding to positions 309 to 379 of B0006991.1, SEQ ID NO: 14) and positions where a forward primer, a reverse primer and a probe were bound to the gene sequence. The forward primer is shown in SEQ ID NO: 7 and starts from the 309th base position of ribonuclease P gene with a length of 19 mer; the reverse primer is shown in SEQ ID NO: 8 and starts from the 379th base position of ribonuclease P gene with a length of 19 mer; the probe is a forward probe shown in SEQ ID NO: 11 and starts from the 330th position of ribonuclease P gene with a length of 21 mer. It could be amplified to form an amplification product with a size of 71 mer using the forward primer and the reverse primer mentioned above.

TABLE 1

| | | | Coverage Rate for |
|---|---|---|---|
| | | | Sequence |
| | | | Comparisons from |
| | | Primer and Probe Sequence | 2018 to October 2023 |
| Sequence ID NO: | Name | 5′→3′ | (NCBI Database) |
| SEQ ID NO: 1 | Influenza A | CTTCTAACCGAGGTCGAAACGTA | 99.41% |
| | Forward Primer 1 | | |
| SEQ ID NO: 2 | Influenza A | CTTCTTACCGAGGTCGAAACGTA | |
| | Forward Primer 2 | | |
| SEQ ID NO: 3 | Influenza A | AGAGCCTCAAGATCTGTGTT | 90.84% |
| | Reverse Primer | | |
| SEQ ID NO: 9 | Influenza A | TCAGGCCCCCTCAAAGCCGAG | 99.72% |
| | Probe | | |
| SEQ ID NO: 4 | Influenza B | TGTCGCTGTTTGGAGACACAATTG | 99.36% |
| | Forward Primer 1 | | |
| SEQ ID NO: 5 | Influenza B | TTCTTTCCCACCGAACCAAC | 98.17% |
| | Reverse Primer 1 | | |
| SEQ ID NO: 6 | Influenza B | TTCTTTCCCACCAAACCAAC | |
| | Reverse Primer 2 | | |
| SEQ ID NO: 10 | Influenza B | AGAAGATGGAGAAGGCAAAGCAGAACTAGC | 97.50% |
| | Probe | | |
| SEQ ID NO: 15 | Influenza A | CATGAGAGCCTCAAGATCTGTGTT | 90.07% |
| | Reverse Primer | | |

### Example 2

The detection method provided by the implementation of the present disclosure was real-time reverse transcription-PCR to realize simultaneous detection of influenza A matrix protein gene, influenza B matrix protein gene and human gene ribonuclease P multiplex qPCR. A reaction mixture included templates (e.g., standards), real-time reverse transcription-PR reagents, primer pairs, and probes whose concentrations are shown in Table 2 below to prepare the reaction mixture. The standards containing the templates included synthetic IVT RNA of influenza A virus, synthetic IVT RNA of influenza B virus, and specimens taken from nasal swabs of normal individuals who were not infected with influenza A virus and influenza B virus.

TABLE 2

| | | | | | | Concentration |
|---|---|---|---|---|---|---|
| Sequence ID NO: | Primer Probe Group | Group 1 | Group 2 | Group 3 | Group 4 | Unit |
| SEQ ID NO: 1 | Forward Primer 1 of Influenza A | 400 | 300 | 200 | 500 | nM |
| SEQ ID NO: 2 | Forward Primer 2 of Influenza A | 400 | 300 | 200 | 500 | nM |
| SEQ ID NO: 3 | Reverse Primer of Influenza A | 800 | 650 | 400 | 500 | nM |
| SEQ ID NO: 9 | Probe of Influenza A | 350 | 300 | 200 | 100 | nM |
| SEQ ID NO: 4 | Forward Primer 1 of Influenza B | 750 | 600 | 400 | 500 | nM |
| SEQ ID NO: 5 | Reverse Primer 1 of Influenza B | 300 | 250 | 150 | 500 | nM |
| SEQ ID NO: 6 | Reverse Primer 2 of Influenza B | 500 | 400 | 250 | 500 | nM |
| SEQ ID NO: 10 | Probe of Influenza B | 300 | 250 | 150 | 100 | nM |
| SEQ ID NO: 7 | Forward Primer of Ribonuclease P | 300 | 250 | 150 | 250 | nM |
| SEQ ID NO: 8 | Reverse Primer of Ribonuclease P | 300 | 250 | 150 | 250 | nM |
| SEQ ID NO: 11 | Probe of Ribonuclease P | 250 | 200 | 100 | 100 | nM |

Temperatures of real-time reverse transcription-PCR used in the present disclosure were designed as follows: (1) maintaining at 50° C. for 5 minutes for reverse transcription stage; (2) maintaining at 95° C. for 30 seconds for RNA deconstruction and enzyme activation stage; (3) performing 45 cycles of PCR stage by denaturizing at 95° C. for 5 seconds and binding and extending at 60° C. for 10 seconds in each cycle, and performing fluorescence detection at the end of each cycle. An overall time of the real-time quantitative-PCR was less than 1 hour.

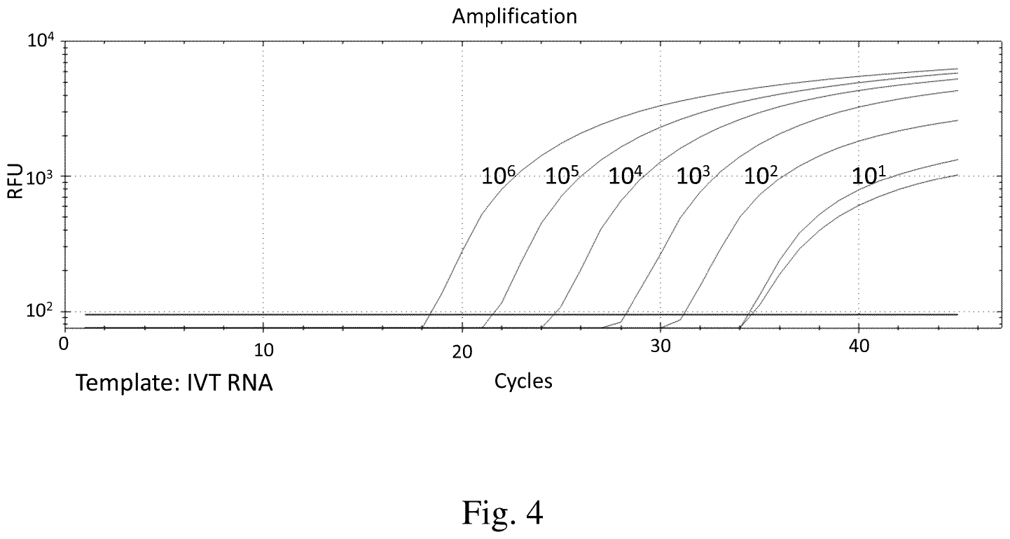

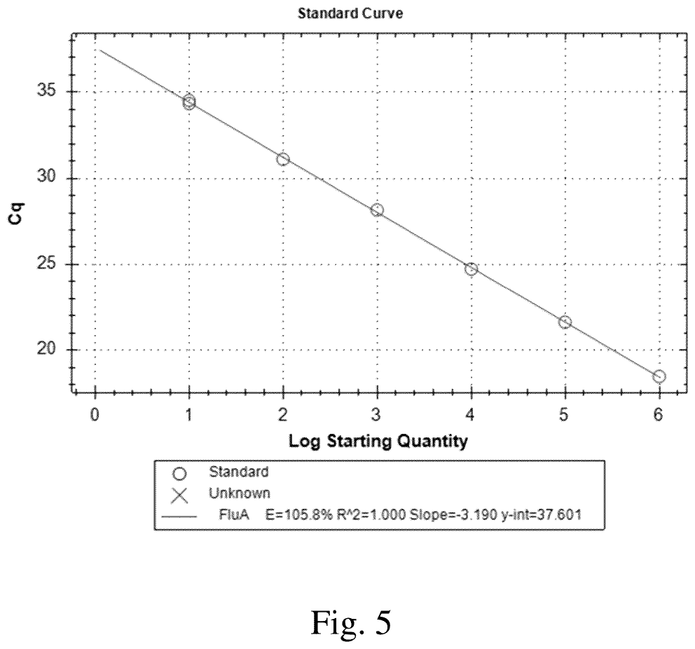

Concentrations of the primers and probes of Group 1 in Table 2 were subjected to the real-time quantitative-PCR. The results showed that FIG. 4 shows different template starting quantities for influenza A virus matrix protein gene according to one embodiment of the present disclosure, and FIG. 5 is a standard curve and an R-squared value of FIG. 4. When influenza A virus matrix protein gene was detected with 10¹-10⁶ cp(copies)/rxn, PCR efficiency (E) was 105.8% and R² was 1.000.

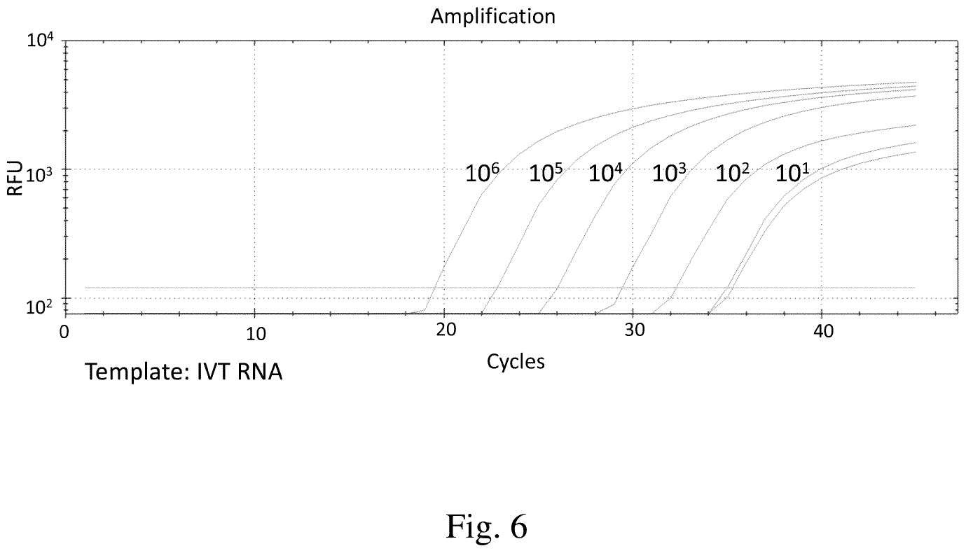

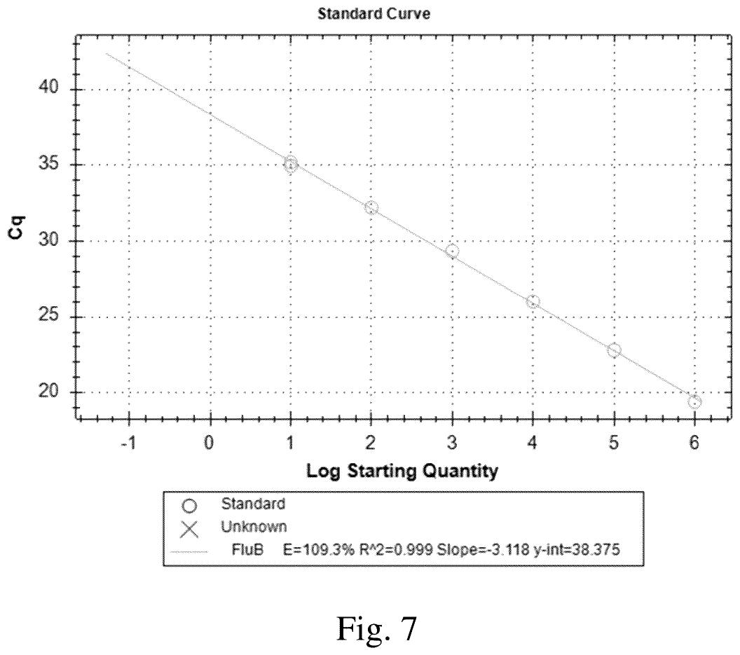

FIG. 6 is a standard curve diagram of different template starting quantities for influenza B virus matrix protein gene according to one embodiment of the present disclosure, and FIG. 7 is a standard curve and an R-squared value of FIG. 6. When influenza B virus matrix protein gene was detected with 10¹-10⁶ cp/rxn, PCR efficiency was 109.3% and R² was 0.999.

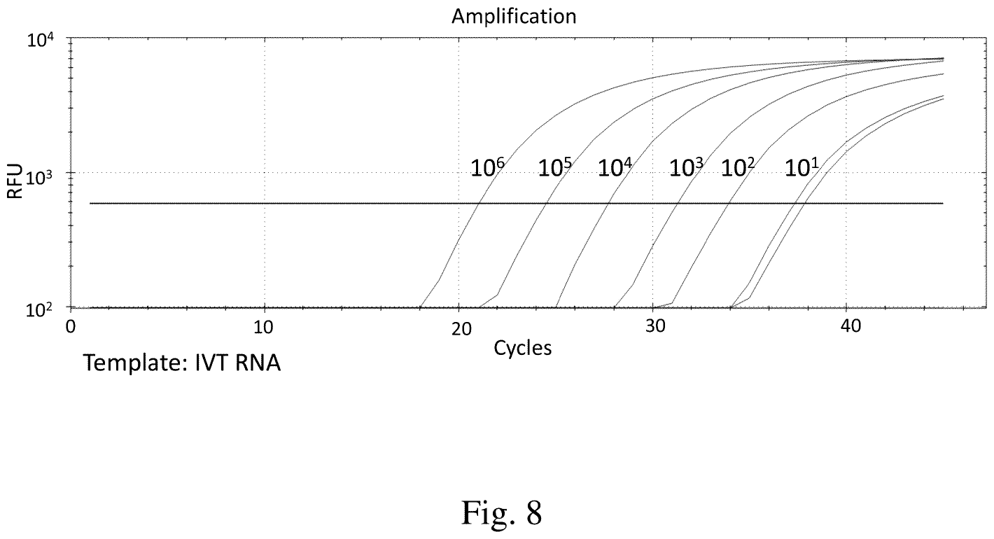

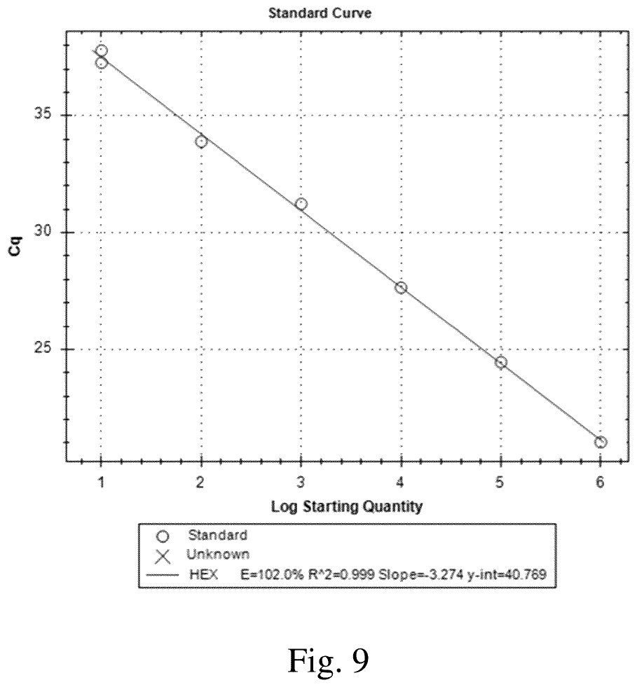

FIG. 8 is a standard curve diagram of different template starting quantities for human gene ribonuclease P according to one embodiment of the present disclosure. FIG. 9 is a standard curve and an R-squared value of FIG. 8. When human gene RNase P was detected with 10¹-10⁶ cp/rxn, PCR efficiency was 102.0% and R² was 0.999.

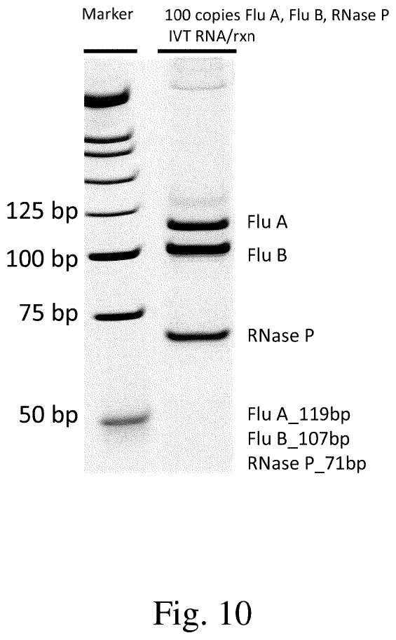

Concentrations of the primers and probes of Group 1 in Table 2 were subjected to the real-time quantitative-PCR. FIG. 10 is an electrophoresis result diagram of detections of influenza A virus matrix protein gene, influenza B virus matrix protein gene and human gene ribonuclease P according to one embodiment of the present disclosure. A template starting quantity of 100 copies was observed by polyacrylamide gel electrophoresis (PAGE). The results showed that product fragments were obvious and there was no strong noise.

### Example 3

The experimental process was the same as in Example 2, and the primer probe groups with different concentrations of Group 1 to Group 4 in Table 2 above were tested. The results are shown in Table 3 below. Influenza A matrix protein gene and influenza B matrix protein gene detected in the present disclosure could withstand interference of 200 nanograms (ng) of human genomic DNA without affecting detection of 100 copies of RNA. A numerical setting of 200 nanograms (ng) of human genomic DNA (gDNA) was based on about 100 ng of human gDNA was collected when simulating the use of a nasopharyngeal swab.

TABLE 3

| | Target | | | | | | | | | | | |
|---|---|---|---|---|---|---|---|---|---|---|---|---|
| | Influenza A (FAM) | | | | Influenza B (ROX) | | | | RNase P (SUN) | | | |
| | Concentration Group of Primer Probe | | | | | | | | | | | |
| | 1 | 2 | 3 | 4 | 1 | 2 | 3 | 4 | 1 | 2 | 3 | 4 |
| 100 cp/rxn | 32.10* | 31.91 | 32.21 | 32.20 | 32.25 | 32.25 | 33.48 | 33.72 | 34.06 | 33.64 | 35.06 | 34.17 |
| 100 cp/rxn + | 34.26 | 33.09 | 33.43 | 33.84 | 34.18 | 33.74 | 34.38 | 34.70 | 23.77 | 23.81 | 24.24 | 24.02 |
| 200 ng gDNA | 34.11 | 34.40 | 33.53 | 34.78 | 33.88 | 33.76 | 33.91 | 36.74 | 23.66 | 23.74 | 24.36 | 24.23 |
| 100 cp/rxn + | 34.26 | 33.09 | 33.43 | 33.84 | 34.18 | 33.74 | 34.38 | 34.70 | 23.77 | 23.81 | 24.24 | 24.02 |
| 300 ng gDNA | 34.11 | 34.40 | 33.53 | 34.78 | 33.88 | 33.76 | 33.91 | 36.74 | 23.66 | 23.74 | 24.36 | 24.23 |

*Cq value (quantification cycle)

Furthermore, the primer probe group of concentrations of Group 1 in Table 2 above was also tested (n=2). Real-time reverse transcription-PCR detection was performed simultaneously using templates of 10 copies and 100 copies with 100 ng, 200 ng, and 300 ng of human gDNA, respectively. The results are shown in Table 4 below. Influenza A matrix protein gene and influenza B matrix protein gene of the detection targets of the present disclosure could withstand interference of 100 to 300 ng of human gDNA without affecting detection of 100 or even 10 copies of RNA.

TABLE 4

| | | Target | | | |
|---|---|---|---|---|---|
| | | Influenza A (FAM) | | Influenza B (ROX) | |
| | | Primer Probe Group | | | |
| Reaction Conditions | | Group 1 | Group 1 | Group 1 | Group 1 |
| 100 cp | Water | 33.34* | 32.50 | 34.27 | 34.09 |
| | 100 ng gDNA | 33.48 | 32.82 | 34.38 | 34.06 |
| | 200 ng gDNA | 35.01 | 32.65 | 34.46 | 34.02 |
| | 300 ng gDNA | 32.73 | 32.77 | 34.18 | 33.84 |
| 10 cp | Water | 43.31 | 37.51 | 39.31 | 39.35 |
| | 100 ng gDNA | 39.13 | 35.88 | 37.94 | 37.10 |
| | 200 ng gDNA | 41.87 | 36.83 | 39.83 | 38.07 |
| | 300 ng gDNA | 37.85 | 36.94 | 37.51 | 36.78 |

*Cq value (quantification cycle)

### Example 4

The experimental process was the same as in Example 2, and the primer probe group of concentrations of Group 1 in Table 2 above was tested. The results are shown in Table 5 below. Sensitivity of a limit of detection (LOD) of simultaneous reaction of the three groups of the primers used in the present disclosure were all less than or equal to 10 copies, in which LOD 95% of influenza A virus was 5 RNA cp/rxn, and LOD 95% of influenza B virus was 4 RNA cp/rxn, and LOD 95% of ribonuclease P was 9 RNA copies/rxn.

TABLE 5

| | | 100 cp/ | 30 cp/ | 10 cp/ | 3 cp/ | | |
|---|---|---|---|---|---|---|---|
| | Target | rxn | rxn | rxn | rxn | NTC* | LOD 95%⁺ |
| Hit rate | Influenza A (FAM) | 20/20 | 20/20 | 20/20 | 13/20 | 0/6 | 5 |
| | Influenza B (ROX) | 20/20 | 20/20 | 20/20 | 17/20 | 0/6 | 4 |
| | RNase P (SUN) | 20/20 | 20/20 | 19/20 | 14/20 | 0/6 | 9 |

*NTC (Non-template control)
⁺probit analysis

### Example 5

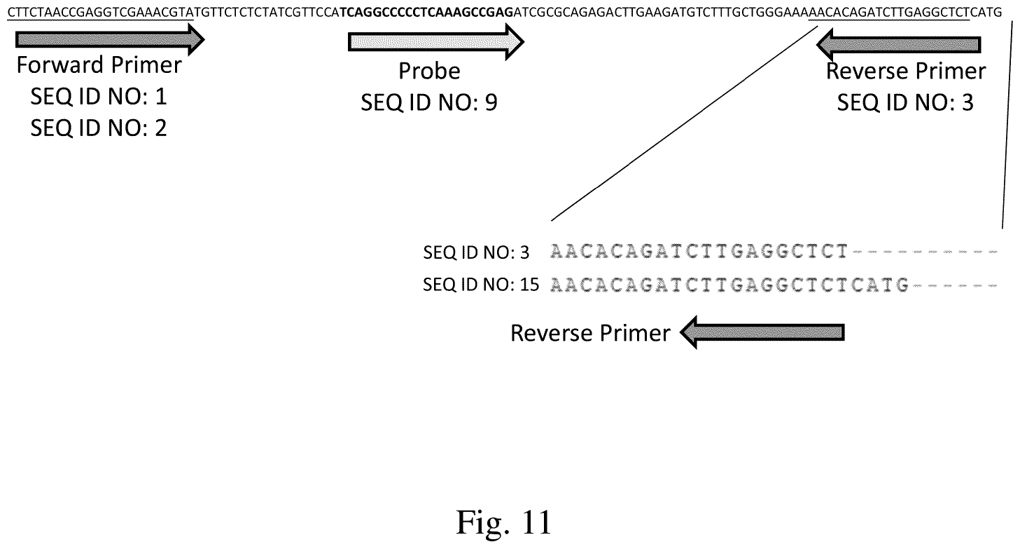

The experimental process was the same as in Example 2, and the primer probe group of concentrations of Group 1 in Table 2 above was tested. The difference was only that SEQ ID NO: 3 was used in one group of the reverse primer of influenza A virus, and SEQ ID NO: 15 was used in another group. Specifically, specificity of SEQ ID NO: 3 was further tested. This experiment was based on the sequence shown in SEQ ID NO: 3, and a similar reverse primer of the primer pair was designed, which was a sequence that was 80% identical to SEQ ID NO: 3. SEQ ID NO: 15 was a derivative sequence of SEQ ID NO: 3, which was modified to increase a length at the 5′ end and still retained the entire sequence of SEQ ID NO: 3 (see FIG. 11). Next, real-time reverse transcription-PCR was performed with this similar primer pair. The detection method is as mentioned above and will not be described in detail here. The results are shown in Table 6 below. Even if SEQ ID NO: 3 had a certain degree of variation, Cq value remained below or equal to 40 cycles (i.e. 35.85) and did not affect detection results of influenza B virus and RNase P when a template quantity was as low as 100 copies. Even under interference of 200 ng human gDNA, influenza A virus, influenza B virus and RNase P could still be successfully detected, and there was not much difference in detection sensitivity from SEQ ID NO: 3.

TABLE 6

| | multiplex qPCR Test | | | | | | |
|---|---|---|---|---|---|---|---|
| | | Target | | | | | |
| | | Influenza A (FAM) | | Influenza B (ROX) | | RNase P (SUN) | |
| | | SEQ ID of Reverse Primer of Influenza A Virus | | | | | |
| Variant | | NO: 3 | NO: 15 | NO: 3 | NO: 15 | NO: 3 | NO: 15 |
| 200 ng gDNA | 100 cp/rxn | 36.57* | 36.25 | 34.12 | 33.09 | 23.02 | 22.62 |
| | | 36.14 | 35.32 | 34.08 | 33.30 | 22.90 | 23.01 |

*Cq value (quantification cycle)

Although the disclosure has been disclosed in the above embodiments, it is not intended to limit the disclosure, and it is to be understood that those skilled in the art can make various changes and modifications without departing from the spirit and scope of the disclosure. The scope of protection of the present disclosure is subject to the definition of the scope of claims.

What is claimed is:

1. A method for detecting influenza A and B viruses, comprising:
providing a sample;
providing a first primer pair, wherein:
a first forward primer of the first primer pair is a nucleotide sequence, its complementary nucleotide sequence or its derivative sequence selected from the group consisting of SEQ ID NO: 1 and a sequence that is from 80% to about 99% identical to SEQ ID NO: 1; and a second forward primer of the first primer pair is a nucleotide sequence, its complementary nucleotide sequence or its derivative sequence selected from the group consisting of SEQ ID NO: 2 and a sequence that is from 80% to about 99% identical to SEQ ID NO: 2; and
a reverse primer of the first primer pair is a nucleotide sequence, its complementary nucleotide sequence or its derivative sequence selected from the group consisting of SEQ ID NO: 3 and a sequence that is from 80% to about 99% identical to SEQ ID NO: 3;
providing a second primer pair, wherein:
a forward primer of the second primer pair is a nucleotide sequence, its complementary nucleotide sequence or its derivative sequence selected from the group consisting of SEQ ID NO: 4 and a sequence that is from 80% to about 99% identical to SEQ ID NO: 4; and
a first reverse primer of the second primer pair is a nucleotide sequence, its complementary nucleotide sequence or its derivative sequence selected from the group consisting of SEQ ID NO: 5 and a sequence that is from 80% to about 99% identical to SEQ ID NO: 5; and a second reverse primer of the second primer pair is a nucleotide sequence, its complementary nucleotide sequence or its derivative sequence selected from the group consisting of SEQ ID NO: 6 and a sequence that is from 80% to about 99% identical to SEQ ID NO: 6; and
performing a polymerase chain reaction on the sample using the first primer pair and the second primer pair, wherein:
when a product obtained through the polymerase chain reaction comprises two with 90 to 130 base pairs, the influenza A and B viruses are detected; or
when the product is not present, the influenza A and B viruses are not detected.

2. The method of claim 1, wherein the sample is nasal mucus, saliva, sputum, blood, urine, feces or a combination thereof.

3. The method of claim 1, wherein the step of performing the polymerase chain reaction on the sample using the first primer pair and the second primer pair comprises: performing the polymerase chain reaction such that the first primer pair amplifies a portion of a sequence of the influenza A virus to SEQ ID NO: 12, and performing the polymerase chain reaction such that the second primer pair amplifies a portion of a sequence of the influenza B virus to SEQ ID NO: 13.

4. The method of claim 1, wherein a concentration ratio of the forward primer to the reverse primer of the first primer pair is from 1:0.5 to 1:2.

5. The method of claim 1, wherein a concentration ratio of the forward primer to the reverse primer of the second primer pair is from 1:1 to 1:2.

6. The method of claim 5, wherein a concentration ratio of the first reverse primer to the second reverse primer of the reverse primer of the second primer pair is from 1:1 to 1:2.

7. The method of claim 1, further comprising:
providing a first probe, which is a nucleotide sequence, its complementary nucleotide sequence or its derivative sequence selected from the group consisting of SEQ ID NO: 9 and a sequence that is from 80% to about 99% identical to SEQ ID NO: 9;
providing a second probe, which is a nucleotide sequence, its complementary nucleotide sequence or its derivative sequence selected from the group consisting of SEQ ID NO: 10 and a sequence that is from 80% to about 99% identical to SEQ ID NO: 10; and
performing the polymerase chain reaction on the sample using the first primer pair, the second primer pair, the first probe and the second probe.

8. The method of claim 7, wherein the polymerase chain reaction is a real-time polymerase chain reaction.

9. The method of claim 1, further comprising:
providing a third primer pair, wherein:
a forward primer of the third primer pair is a nucleotide sequence, its complementary nucleotide sequence or its derivative sequence selected from the group consisting of SEQ ID NO: 7 and a sequence that is from 80% to about 99% identical to SEQ ID NO: 7;
a reverse primer of the third primer pair is a nucleotide sequence, its complementary nucleotide sequence or its derivative sequence selected from the group consisting of SEQ ID NO: 8 and a sequence that is from 80% to about 99% identical to SEQ ID NO: 8; and
performing the polymerase chain reaction on the sample using the first primer pair, the second primer pair and the third primer pair, wherein when the product obtained through the polymerase chain reaction further comprises one with 60 to 80 base pairs, the sample is confirmed from a human.

10. The method of claim 9, wherein the step of performing the polymerase chain reaction on the sample using the first primer pair, the second primer pair and the third primer pair comprises: performing the polymerase chain reaction such that the third primer pair amplifies a portion of a sequence of ribonuclease P of the human to SEQ ID NO: 14.

11. The method of claim 9, further comprising:
providing a first probe, which is a nucleotide sequence, its complementary nucleotide sequence or its derivative sequence selected from the group consisting of SEQ ID NO: 9 and a sequence that is from 80% to about 99% identical to SEQ ID NO: 9;
providing a second probe, which is a nucleotide sequence, its complementary nucleotide sequence or its derivative sequence selected from the group consisting of SEQ ID NO: 10 and a sequence that is from 80% to about 99% identical to SEQ ID NO: 10;
providing a third probe, which is a nucleotide sequence, its complementary nucleotide sequence or its derivative sequence selected from the group consisting of SEQ ID NO: 11 and a sequence that is from 80% to about 99% identical to SEQ ID NO: 11; and
performing the polymerase chain reaction on the sample using the first primer pair, the second primer pair, the third primer pair, the first probe, the second probe and the third probe.

12. A kit for detecting influenza A and B viruses, comprising:
a first primer pair, wherein:
a first forward primer of the first primer pair is a nucleotide sequence, its complementary nucleotide sequence or its derivative sequence selected from the group consisting of SEQ ID NO: 1 and a sequence that is from 80% to about 99% identical to SEQ ID NO: 1; and a second forward primer of the first primer pair is a nucleotide sequence, its complementary nucleotide sequence or its derivative sequence selected from the group consisting of SEQ ID NO: 2 and a sequence that is from 80% to about 99% identical to SEQ ID NO: 2; and
a reverse primer of the first primer pair is a nucleotide sequence, its complementary nucleotide sequence or its derivative sequence selected from the group consisting of SEQ ID NO: 3 and a sequence that is from 80% to about 99% identical to SEQ ID NO: 3; and
a second primer pair, wherein:
a forward primer of the second primer pair is a nucleotide sequence, its complementary nucleotide sequence or its derivative sequence selected from the group consisting of SEQ ID NO: 4 and a sequence that is from 80% to about 99% identical to SEQ ID NO: 4; and
a first reverse primer of the second primer pair is a nucleotide sequence, its complementary nucleotide sequence or its derivative sequence selected from the group consisting of SEQ ID NO: 5 and a sequence that is from 80% to about 99% identical to SEQ ID NO: 5; and a second reverse primer of the second primer pair is a nucleotide sequence, its complementary nucleotide sequence or its derivative sequence selected from the group consisting of SEQ ID NO: 6 and a sequence that is from 80% to about 99% identical to SEQ ID NO: 6.

13. The kit of claim 12, further comprising:
a first probe, which is a nucleotide sequence, its complementary nucleotide sequence or its derivative sequence selected from the group consisting of SEQ ID NO: 9 and a sequence that is from 80% to about 99% identical to SEQ ID NO: 9; and
a second probe, which is a nucleotide sequence, its complementary nucleotide sequence or its derivative sequence selected from the group consisting of SEQ ID NO: 10 and a sequence that is from 80% to about 99% identical to SEQ ID NO: 10.

14. The kit of claim 12, further comprising:
a third primer pair, wherein:
a forward primer of the third primer pair is a nucleotide sequence, its complementary nucleotide sequence or its derivative sequence selected from the group consisting of SEQ ID NO: 7 and a sequence that is from 80% to about 99% identical to SEQ ID NO: 7; and
a reverse primer of the third primer pair is a nucleotide sequence, its complementary nucleotide sequence or its derivative sequence selected from the group consisting of SEQ ID NO: 8 and a sequence that is from 80% to about 99% identical to SEQ ID NO: 8.

15. The kit of claim 14, further comprising:
a third probe, which is a nucleotide sequence, its complementary nucleotide sequence or its derivative sequence selected from the group consisting of SEQ ID NO: 11 and a sequence that is from 80% to about 99% identical to SEQ ID NO: 11.
